(19) United States
(12) Patent Application Publication
(10) Pub. No.: US 2015/0283269 A1
(43) Pub. Date: Oct. 8, 2015

(54) RECONSTITUTED HUMAN IMMUNE SYSTEM IN A PATIENT DERIVED XENOGRAFT MOUSE MODEL

(71) Applicant: Crown Bioscience Inc., Santa Clara, CA (US)

(72) Inventors: Xiaoyu AN, Beijing (CN); Henry LI, Santa Clara, CA (US)

(73) Assignee: Crown Bioscience Inc., Santa Clara, CA (US)

(21) Appl. No.: 14/245,999

(22) Filed: Apr. 4, 2014

(51) Int. Cl. A61K 49/00; C07K 16/28; A01K 67/027; A61K 33/24

(52) U.S. Cl. CPC A61K 49/0008; A61K 33/24; C07K 16/2863; A01K 67/0271; A01K 2227/105; A01K 2267/03

(57) ABSTRACT

The present teachings relate to methods of screening for a therapeutic agent, selecting a treatment and monitoring a treatment for a human disease or infection and methods for producing a mouse model for human disease or infection wherein the mouse has a functioning human immune system. The method includes administering a test substance to an immunocompromised NOD/SCID mouse with a reconstituted human immune system and is also engrafted with a substance containing a diseased or infectious cell derived from a human diseased or infected patient and a step of assessing improvement in the disease or infection of the mouse and/or to monitor a side effect of the test substance.

### FIELD

The present teachings relate to the use of patient derived xenographic mouse models which include a reconstituted human immune system for evaluating new chemotherapeutics and the treatment of patients.

### BACKGROUND

The current mouse models for human disease are constructed by engrafting a human disease cell into an immunocompromised mouse. The limitation with such a model is that evaluation of new drugs, determining a treatment regime or screening a patient for disease response occurs in the absence of a functional immune system. Too, because there are differences between the mouse and human immune systems it has been shown that the mouse does not always resemble a human disease state.

Therefore, there remains a need to advance the investigation of new therapeutic agents treatment regimens or screening a patient for disease response using animal models. The disclosed animal model can, by engraftment, replicate human disease and also the human immune system. In particular, such a disease model system can rapidly advance the search for new treatments and treatment strategies against tumors, cancers and infections. The use of animal models for screening new treatments and optimizing treatment strategies would be most advantageous to advancing survival of patients afflicted with disease or infection as well as to advance the understanding of new therapeutics to augment the human immune response in treating human disease and infection.

### SUMMARY

In one embodiment disclosed is a method for screening for a therapeutic agent for a diseased human, the method having one skilled in the art of studying animal models for disease to administer an agent to a first immunocompromised NOD/SCID mouse, wherein the mouse i) is immunodeficient for a mouse immune system, ii) is engrafted with human stem cells containing human CD34+ cells, wherein the CD34+ cells are isolated from fetal liver, peripheral blood or umbilical cord blood cells, and iii) is engrafted with a substance derived from a diseased human containing a diseased cell or tissue derived from a human diseased patient or is engrafted with a substance derived from a second immunocompromised NOD/SCID mouse engrafted with a substance derived from a diseased human containing a diseased cell or tissue derived from a human diseased patient; and determining whether the agent positively impacts the amount of the human disease in the human disease-infected mouse.

In further embodiments, the mouse is immunocompetent for a human immune system, wherein the mouse further displays the histopathology of the disease of the human disease patient and wherein the mouse displays the genetic profile of the diseased cell derived from the human disease patient. In yet further embodiments, the agent is a drug, or a biologic and the human disease is selected from a malignant neoplasia and an infection and the diseased cell derived from a human diseased patient is selected from an epithelial cell, a bacterium, a parasite, a virus, a piron or a microbacterium.

In still further embodiments, the amount of the human disease impacted by the agent is determined by monitoring at least one of: tumor size, tumor pathology, human cell markers within the tumor, prolonged survival, no disease relapse, and immune response and combinations thereof. In yet another embodiment the biologic is selected from an antibody, a vaccine, an allergenic, a somatic cell, a gene therapy, a tissue, a recombinant therapeutic protein or a living cell used as a therapeutic. In further embodiments, the malignant neoplasia is selected from a non-small cell lung cancer, a breast cancer, a gastric cancer and a colon cancer and wherein the tumor pathology is determined by staining for hematopoiesis cells. Further, the hematopoiesis cells are selected from lymphocytes, dentritic cells, macrophage cells, monocytes, MHC-II cells, pre-B cells, B-cells, T-cells, and natural killer cells and can be present in the tumor pathology which would indicate the presence of a human tumor infiltrate lymphocyte (TIL) cell, and wherein the tumor pathology indicates the presence of an inflammatory cell is at least one of a CD4, CD11b, CD14 and CD33 cell and combinations thereof. In further embodiments the cells resulting from hematopoiesis can have detectable cell markers, wherein the human cell markers within the tumor are at least one of CD11c in Dentritic Cells; HLA-DR in MHC-II Cells; CD19 in Pre-B cells; CD20 in B-Cells; CD4 in T-Cells; CD56 in NK Cells; CD123 in cells with IL-3 receptor and CD25 with the IL-2 receptor, each as detectable markers of lymphocyte activation for many immune cells. The immune cells include T cells, activated B cells, some thymocytes, and myeloid precursors. In yet another embodiment, the epithelial cell is from a non-small cell lung cancer, a breast cancer, a gastric cancer or a colon cancer.

In still yet another embodiment, the second immunocompromised NOD/SCID mouse is engrafted with a substance derived from a third or a fourth or fifth or sixth diseased mouse engrafted with a substance containing a diseased cell or tissue derived from the same or a different human diseased patient or from a second different diseased mouse engrafted with a substance derived from a different diseased mouse engrafted with a substance containing a diseased cell or tissue derived from an engrafted diseased mouse having the human disease.

In yet another embodiment, disclosed is a method of selecting or optimizing a method of treating a patient from whom tumor cells in the peripheral blood are derived from an immunocompromised NOD/SCID mouse engrafted with human stem cells containing human CD34+ cells, wherein the CD34+ cells are isolated from fetal liver, peripheral blood or umbilical cord blood cells and engrafted with a substance derived from: i) a diseased human containing a diseased cell or tissue derived from a human diseased patient, or ii) a diseased mouse containing a diseased cell or tissue derived from at least a second or a third immunocompromised NOD/SCID diseased mouse engrafted with a substance derived from a second diseased mouse containing a diseased cell or tissue derived from a human diseased patient, and a) providing the mouse with a treatment for the tumor, and b) assessing an improvement and/or a side effect caused by the treatment of the tumor in the mouse.

In still yet another embodiment, disclosed is an immunocompromised NOD/SCID diseased mouse comprising a functioning human immune system and a replicate pathology of a human disease, wherein the mouse is a xenografted mouse.

In yet another embodiment, disclosed is a method of producing a mouse having a human immune system, comprising engrafting a substance with human stem cells containing human CD34+ cells, wherein the CD34+ cells are isolated from fetal liver, peripheral blood or umbilical cord blood cells into a NOD/SCID mouse and raising the mouse. The mouse can be further engrafted with a substance derived from a diseased human containing a diseased cell or tissue derived from a human diseased patient, or engrafting the mouse with a substance derived from a diseased mouse containing a diseased cell or tissue derived from at least a second or a third immunocompromised NOD/SCID diseased mouse engrafted with a substance derived from a second diseased mouse containing a diseased cell or tissue derived from a human diseased patient and raising the mouse.

In still yet another embodiment, disclosed is a method of producing a mouse having a human immune system and a human disease, comprising a). engrafting a substance with human stem cells containing human CD34+ cells, wherein the CD34+ cells are isolated from fetal liver, peripheral blood or umbilical cord blood cells into a NOD/SCID mouse, b). i) engrafting the mouse with a substance derived from a diseased human containing a diseased cell or tissue derived from a human diseased patient, or ii) engrafting the mouse with a substance derived from a diseased mouse containing a diseased cell or tissue derived from at least a second or a third immunocompromised NOD/SCID diseased mouse engrafted with a substance derived from a second diseased mouse containing a diseased cell or tissue derived from a human diseased patient and raising the mouse.

In yet another embodiment, disclosed is a method for identifying an agent for treating a human malignant neoplasia in a mouse model, the method having: a) administering an agent to a first immunocompromised mouse, wherein the mouse i) is immunodeficient for a mouse immune system, ii) is engrafted with human stem cells containing human CD34+ cells, wherein the CD34+ cells are isolated from fetal liver, peripheral blood or umbilical cord blood cells, and

iii) is engrafted with a substance derived from a diseased human containing a diseased cell or tissue derived from a human diseased patient or is engrafted with a substance derived from a second immunocompromised NOD/SCID mouse engrafted with a substance derived from a diseased mouse containing a diseased cell or tissue derived from a human diseased patient; and b) determining whether the agent positively impacts the amount of the human disease in the human disease-infected mouse. The method can further have c) a step of monitoring a side effect of the test substance in the mouse by examining the peripheral blood collected from the mouse in step b) and/or step c), and wherein the method can reproduce in the mouse the pathology of the malignant neoplasia in the patient from whom the substance is derived and then selectively expanded the human malignant neoplasia cells within the mouse by engrafting the mouse with cells or tissues derived from a diseased human patient or cells or tissues derived from a mouse previously engrafted with cells or tissues derived either from a diseased human patient or cells or tissues derived from a diseased mouse which has the same disease as the diseased human patient. The patient derived xenograft (PDX) mouse can further selectively expand malignant neoplasia cells or tissues from a non-small cell lung cancer, breast cancer, gastric cancer or colon cancer and serve as an experimental mouse model for a cancer and the mouse further exhibits at least one of an enlarged spleen, tumor infiltrate lymphocytes or the presence of inflammatory cells and the lymphocytic cells are present in at least one of tumor or spleen.

In still yet another embodiment disclosed is a method of producing a mouse having human malignant neoplasia cells, comprising engrafting a substance containing a malignant neoplasia cell or tissue derived from a human malignant neoplasia patient into a non-adult immunocompromised NOD/SCID mouse and raising the mouse.

In yet another embodiment disclosed is a method of producing a mouse having human malignant neoplasia cells, comprising one or more repeats of a step of engrafting a substance containing a malignant neoplasia cell or tissue derived from the mouse obtained by engrafting a substance containing a malignant neoplasia cell or tissue derived from a human malignant neoplasia patient into a non-adult immunocompromised NOD/SCID mouse into a different non-adult immunocompromised NOD/SCID mouse and raising the mouse.

In any of the embodiments described above, the methods can include examining the peripheral blood collected from the mouse about four weeks after engraftment, or at any time after administering to the engrafted mouse a treatment for a tumor or disease or to determine if a treatment has a toxicological affect or if a treatment has no therapeutic impact on the tumor or disease load in the mouse's peripheral blood, bone marrow or spleen or other organs such as the head, kidney, gastrointestinal tract, colon, or lung. The methods can also include examining the engrafted cells for production of at least one of GM-CSF and IL-3 as well as chemokines and cytokines.

In any of the embodiments described above, the methods by which a mouse is engrafted with malignant neoplasia cells or tissue derived from another engrafted mouse reproduces the pathology of malignant neoplasia in the mouse of the patient from whom the substance containing a malignant neoplasia cell or tissue derived from a human malignant neoplasia patient is derived. The engrafted mouse also reproduces the phenotype and genotype of the malignant neoplasia cells in the mouse of the patient from whom the substance containing a malignant neoplasia cell derived from a human malignant neoplasia patient is derived. Additionally, the engrafted cells can be selectively expanded human tumor cells, which are obtained by one or more repeats of a step of engrafting a malignant neoplasia cell or tissue derived from a human malignant neoplasia patient into a non-adult immunocompromised NOD/SCID mouse and raising the mouse. Such a mouse is known as a malignant neoplasia mouse, can serve as an experimental model and exhibits at least one of an enlarged spleen or the presence of tumor cells. The tumor cells are present in at least one of bone marrow or a peripheral organ such as spleen or blood. Additionally, the malignant neoplasia mouse has a malignancy of hematopoiesis or a cancer and the hematopoiesis cells are myelopoiesis cells or lymphopoiesis cells and the cancer is a non-small cell lung cancer, a breast cancer, a gastric cancer or a colon cancer.

In any of the embodiments described above, the hematopoiesis or cancer cell or cancer tissue can be a cell or tissue, extracted from a tissue in the patient's bone marrow, spleen or blood or tumor tissue and the extracted cell is obtained by Ficoll® column separation (Ficoll Paque™ PLUS solution, GE Healthcare Bio-Sciences, Piscataway, N.J.) or the use of a CD34 MicroBead Kit (Miltenyl Biotec, Inc., Auburn, Calif.) according to manufacturer's instructions. The engraftment results in a high percentage of hematopoiesis or cancer cells seen in bone marrow as well as in peripheral organs such as at least one of the blood and spleen.

### BRIEF DESCRIPTION OF THE DRAWINGS

FIG. 1. Illustration of the method for engrafting a NOD/SCID mouse with CD34+ human stem cells to reconstitute a human immune system and also engrafting patient derived tumor cells engrafted (PDX) into the same mouse. Also listed are immune cells and some of the surface proteins detectable and expressed by the reconstituted immune system.

FIG. 2. 2A: Graphical illustration of the increasing percentage of engrafted humane cells found in mouse circulating peripheral blood following inoculation; 2B: Graphical illustration of the different cell lineages a termination of the PDX mouse; 2C: Representative FACS analysis of human cell surface proteins in peripheral blood of the PDX mouse; 2D-2F: Bar graft illustrations of human immune cell lineages in PDX mouse D: peripheral blood; E: bone marrow; F: spleen.

FIG. 3. Graphical comparison of the tumor growth of non-small cell lung cancer in a PDX mouse with a reconstituted human immune system (HuMice) vs. a negative control mouse (Saline).

FIG. 4. A: Graphical comparison of the tumor growth of non-small cell lung cancer in a PDX mouse with a reconstituted human immune system plus antibiotic therapy treatment (HuMice+Cetuximab) vs. a PDX mouse with a reconstituted human immune system without antibiotic therapy treatment (Saline); B: Graphical comparison of the tumor growth of non-small cell lung cancer in a PDX mouse without a reconstituted human immune system plus antibiotic therapy treatment (Saline+Cetuximab) vs. a PDX mouse without a reconstituted human immune system without antibiotic therapy treatment (Saline+Saline).

FIG. 5. A: Bar graft illustration of the various human cell lineages that infiltrated a reconstituted PDX mouse having a human tumor; B: Bar graft illustration of human immune cell lineages in reconstituted PDX diseased mouse tumor; C: Photo comparisons of microscopic sections of tumor and spleen samples stained with anti-CD45 antibody stain in reconstituted (HuMice™ mouse) and immunocompromised (NOD/SCID) PDX mice.

FIG. 6. A-C: Photos and bar graft illustrating Natural Killer (NK) cell activation in reconstituted PDX GA0055 HuMice™ mouse. A: photo of microscopic (40λ10) sections of H&E cells staining positive for NK cell activation: B: HER2 immunohistochemical staining of NK cell activation, (Score 3+); C: Bar graft illustrating experimental level of NK cell activation.

FIG. 7. Comparisons of reconstituted and immunocompromised mice after transplantation with over-expressing gastric cancer cells and chemotherapeutic treatment or no treatment. A: Reconstituted mice with and without Herceptin treatment; B: NOD/SCID mice with and without Herceptin treatment; C: Reconstituted mice with and without Cisplantin treatment; D: NOD/SCID mice with and without Cisplantin treatment.

### DESCRIPTION OF VARIOUS EMBODIMENTS

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting.

As used in this specification and the appended claims, the singular forms “a”, “an”, and “the” include plural references unless the context clearly dictates otherwise. Thus, for example, references to “a leukemic” includes one or more leukemic cells, and/or compositions of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Any methods and materials similar or equivalent to those described herein can be used in the practice or testing of the invention, as it will be understood that modifications and variations are encompassed within the spirit and scope of the instant disclosure. All publications mentioned herein are incorporated herein by reference in their entirety.

Reference will now be made to various embodiments, examples of which are illustrated in the accompanying drawings.

The present teaching can be applicable to Patient Derived Xenograft (PDX) models known to be closely reflective of tumors or infections in patients for both their histopathological and genetic profiles (Ding, L., et al., 2010 Nature 464:999-1005; Marangoni, E., et al., 2007 Clin. Cancer Res. 13:3989-3998). PDX mice have become widely accepted as the model of choice to evaluate anticancer agents for enhancing predictive power. However, current PDX models (HuPrime® 1.0/2.0 mice, Crown Biosciences, Inc., Santa Clara, Calif.) still have limitations. One such limitations is that tumor growth occurs in an environment lacking functional immunity, in particular that of human immunity.

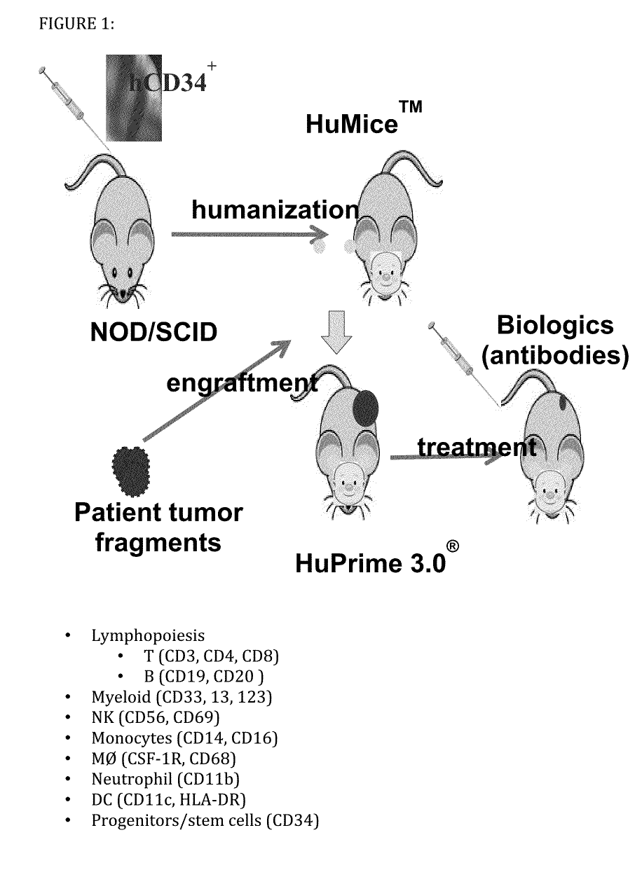

Disclosed are PDX mice having a functioning human immune response. The human immune functions have been successfully reconstituted in the same types of immunodeficient mice that were also used for engrafting PDX. The mouse having a humanized and functional human immune system was established normally through engraftment of human stem cells containing human CD34⁺ cells isolated from fetal liver, peripheral blood or umbilical cord blood (UCB). Described is a single mouse having both an engrafted human derived immune system and engrafted patient tumor cells or having an infectious disease to more closely replicate the patient environment (FIG. 1). The new mouse model has been shown to be an improved predictive experimental model of disease progression, prognosis as well as for screening new therapeutics, monitoring therapeutic response and designing therapeutic treatment protocols.

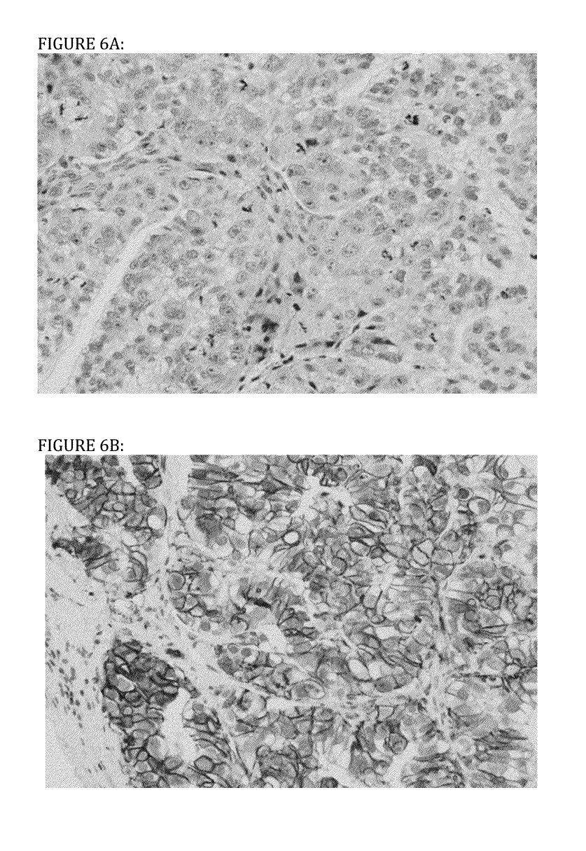

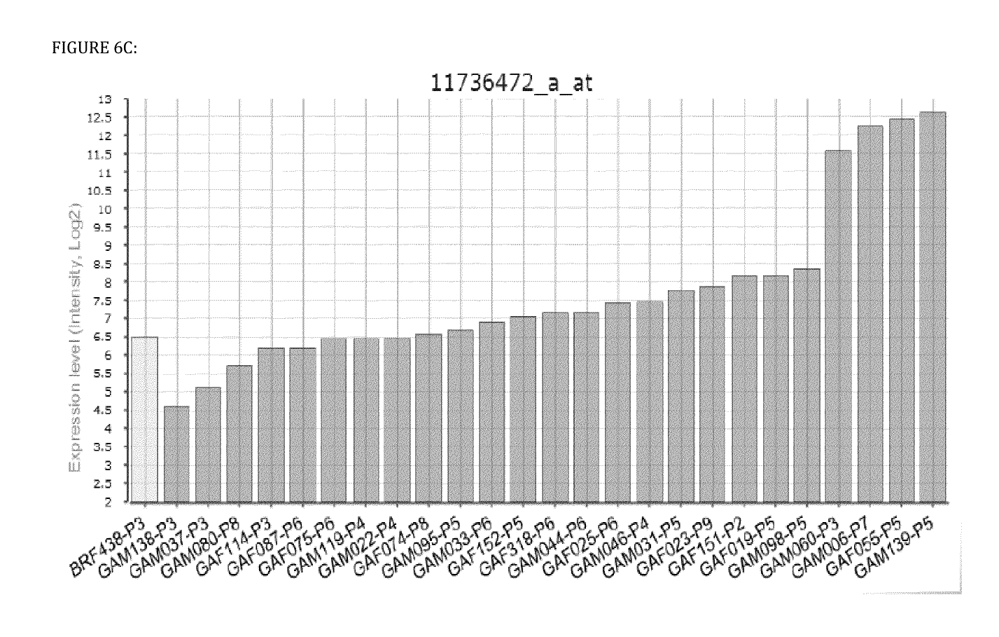

In the present study, NOD/SCID mice were engrafted with CD34⁺ UCB cells (hematopoietic stem cell enriched population) and demonstrated full reconstitution of human hemopoiesis, including myelopoiesis, lymphopoiesis (both T and B lineages) (FIGS. 1A, 1B), growth of dentritic cells (DCs), macrophage and monocytes, and growth of natural killer cells (NKs) were detected by FACS (FIG. 1C). Human immune cell lineages were also identified in peripheral blood (PB), bone marrow (BM) and spleen (FIGS. 1D-1F, respectively). Taken alone or together we have demonstrated the existence of normal human immune functions in the reconstituted mice (HuMice™ mouse model), including production of human cytokines such as GM-CSF and IL-3 (FIGS. 6A-6B).

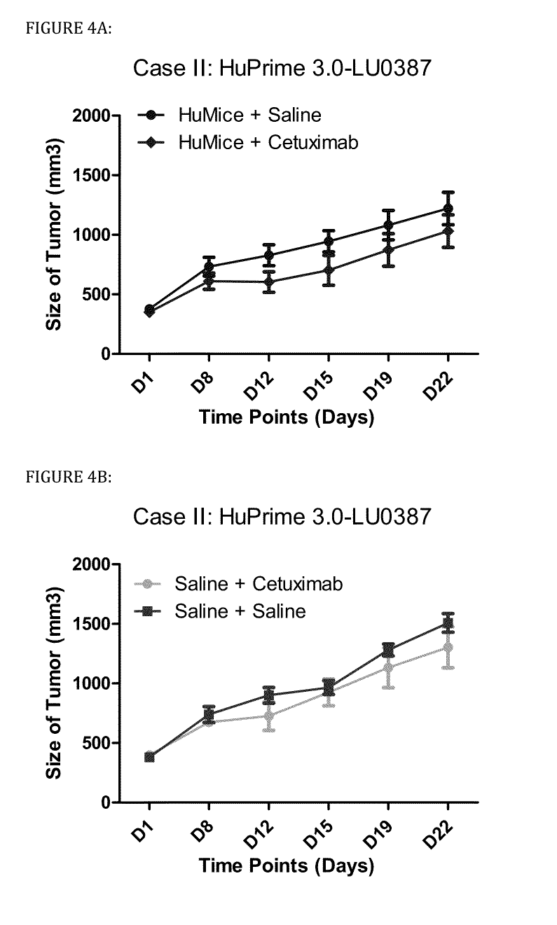

The HuMice™ mouse model is also useful when engrafted with a human tumor cells, tissue or an infectious disease/organism. Using a previously established non-small cell lung cancer PDX model (HuPrime® mouse model, Crown Bioscience) for non-small cell lung cancer, LU2503 (Yang, M., et al., 2013 Int. J. Cancer, E74-E84, September 5. doi: 10.1002/ijc.27813. [Epub ahead of print], FIG. 3) and LU0387 (FIGS. 4A and 4B) were separately engrafted into the CD34⁺ UCB reconstituted NOD/SCID mice.

As depicted in FIG. 3, the NSCLC tumor appears to grow at a slower rate in the immune reconstituted HuMICE™ model showing that not only has the human immune system, as a xenograft, has been established but that the immune system appears to slow tumor growth in vivo in the HuMice™ model. The results demonstrated that the tumor in the normal human immune system grew well, though potentially at slightly slower growth kinetics than the tumors in mice without reconstitution.

Further preliminary evaluations were also undertaken to evaluate the new PDX model (HuPrime™ 3.0 mouse model) by treating the new human immunocompetent PDX mice with antibody therapeutics. Initial data showed that the presence of human immunity, such as human NK-mediated antibody-dependent cell-mediated cytotoxicity (ADCC), had a slightly enhanced anti-tumor effect of the antibody drug Cetuximab in the HuPrime™ 3.0 mice (HuMice™ mouse+PDX for NSCLC+Cetuximab or Saline; FIG. 4A). Further work to assess the role of the reconstituted immune system's function in concert with a biologic drug effect was also performed. Standard NOD/SCID mice, PDX for NSCLC were treated with either saline or Cetuximab. As shown in FIG. 4B efficacy of the monoclonal antibody Cetuximab was virtually non-existent when the mouse model lacked a reconstituted human immune system including a functioning ADCC mechanism. The ADCC mechanism is consider an active participant in therapeutic monoclonal antibody efficacy (Clynes, R A, et al., 2000, Nat. Med. 6(4):443-6. doi:10.1038/74704. While not wishing to be bound by any theory, it could be that the HuPrime™ 3.0 mouse model, having a reconstituted human immune system could represent a more predictive model of drug efficacy than those mouse model systems without functional human immunity.

In yet another example of the utility of the HuPrime™ 3.0 mouse model (PDX human immunocompetent NOD/SCID mouse with CD34⁺ hSC+PDX gastric tumor), patient cells derived from a human patient diagnosed with over-expressing gastric cancer, GA 0055, were engrafted into HuPrime™ 3.0 mice. Table 1 lists the tumor pathology and Table 2 the genotype for GA 0055.

TABLE 1
Summary of GA 0055 patient information

| | | |
|---|---|---|
| Patient | Female, 69-yrs old | |
| Tumor stage/ | T?N3M0 IV/II | |
| Grade | | |
| Pathology | Clear cell adenocarcinoma of anterior | Pathology QC: |
| | wall of gastric antrum, ulcerative | P2- Poorly |
| | type, moderately differentiated. | differentiated |
| | Regional LN: LN of lesser curvature | adenocarcinoma, |
| | (2/6), LN of greater curvature (2/6), | with part of |
| | LN of the eighth group (1/1). IHC | mucinous |
| | results: HER-1(−), HER-2(+), | adenocarcinoma, |
| | p53(+25~50%), p170(focus+), | P8- Papillary |
| | Ki-67(+<10%), VEGF(++), | adenocarcinoma |
| | Top-IIa(+<1%), p16(++). | |
| Mutation | U219 - P5 | |
| | SNP - P5 | |

TABLE 2
Genetic mutation status

| Gene Symbol | Value |
|---|---|
| AKT1 \| Exon3 | WT |
| BRAF \| Exon15 | WT |
| EGFR \| Exon18; 19; 20; 21 | WT |
| KRAS \| Exon2; 3; 4 | WT |
| MAPK1 \| Exon2; 8 | WT |

The HuPrime™ 3.0 mice engrafted with GA 0055 were divided into six groups, four mice each. Three groups were engrafted with UCB human stem cells and three groups were not. Preliminary data (not shown) indicated slightly slower tumor growth of GA 0055 in the UCB groups verses the three groups without UCB engraftment. The groups were then evaluated for treatment response to 6 mg/kg body weight of Herceptin or 5 mg/kg body weight of Cisplatin. FIG. 6A provides a hematoxylin and eosin (H&E) stained cross-section of GA 0055 tumor cross-sections (40×10) treated with Cisplatin indicates Cisplatin is effective against GA 00555 while FIG. 6B is an immunohistorchemical stained section of GA 0055 treated with HER2. The extensive black staining of GA 0055 with H&E stain in FIG. 6B indicates that GA 0055 is non-responsive to Herceptin in the absence of human immunity. This is yet another example of the role of human immunity in providing the ADCC mechanism to facilitate treatment with monoclonal antibody therapeutics.

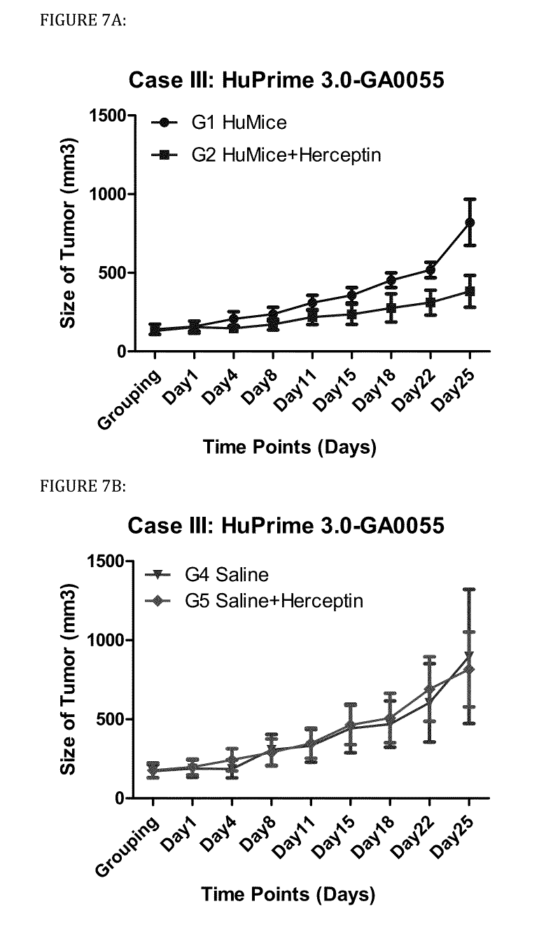

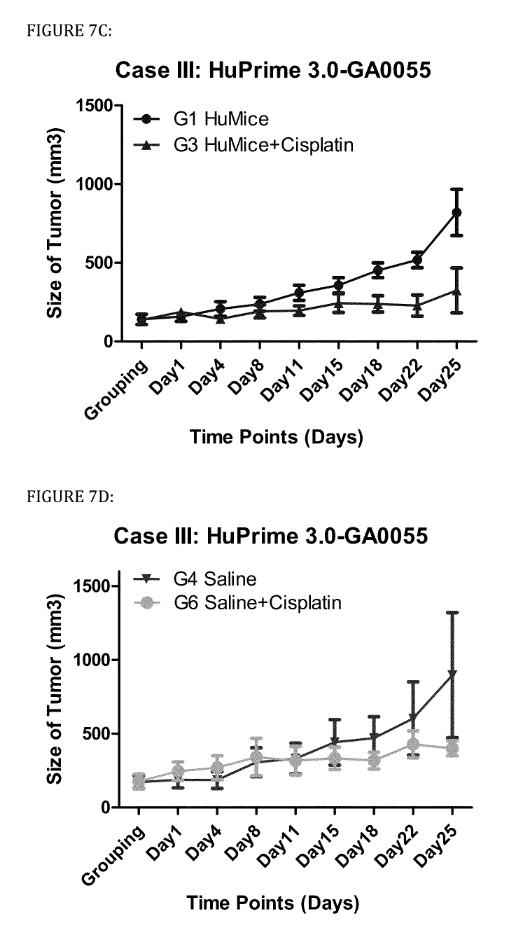

To further illustrate the ADCC mechanism and the response of GA 0055 to treatment HuMice™ mice (reconstituted human immunocompetent) were engrafted with GA 0055 and then treated with or without Herceptin. Likewise, NOD/SCID (immunodeficient) mice were similarly engrafted and treated with Herceptin. As illustrated in FIG. 7A, treatment with Herceptin slowed tumor growth when human immune function was present while there is no impact in the NOD/SCID mice, FIG. 7B. As seen in FIG. 7C, treatment of PDX GA 0055 HuMice™ mice with the chemotherapy drug Cisplatin also slowed tumor growth in the presence of Cisplatin and similar results were achieved in NOD/SCID mice also treated with Cisplatin (FIG. 7D). These results substantiate that Cisplatin is not dependent on a functioning human immune system, unlike Herceptin.

In one embodiment disclosed is a newly established model of human immunity in NOD/SCID mice by engrafting CD34+ human stem cells. The model reflects aspects of human immunity and the immune cells are able to infiltrate tumors as well as assist in the ADCC mechanism of action when a monoclonal antibody is administered to treat a PDX mouse model. Several unique characteristics of this model distinguished it from many other reported models including; A more predictive mouse model, a full reconstitution of human hemopoiesis, including myelopoiesis, lymphopoiesis (both T and B lineages), growth of dentritic cells (DCs), macrophage and monocytes, and growth of natural killer cells (NKs), normal immune functions in the reconstituted mice (HuMICE™ mice), and successfully engrafted a previously established non-small cell lung cancer PDX (HuPrime® mouse) model (LU2503 and LU0387) into the CD34⁺ UCB reconstituted NOD/SCID mice suggesting the possibility to engraft diseased patient cells from a mouse xenograft into a HuPrime® mouse thus establishing a mouse model having a reconstituted immune system and good tumor growth thus permitting a more predictive model for the roll of reconstituted immunity played in examining a biologic drug's effect on tumor growth, designing treatment strategies and evaluating side-effects of a therapeutic or biologic treatment.

### GENERAL DEFINITIONS

The phrase “amount of the human disease impacted by the agent” as used herein refers to at least one of the growth of the tumor, weight of the tumor, size of the tumor, weight of the rodent who received the agent as a treatment or screen for a treatment for a human disease and the like as a way to access the efficacy of a human disease treatment agent. As used herein “agent” refers to a biologic or a chemical treatment for a human disease or infection.

The phrase “about four weeks old” as used herein refers to a non-mature rodent being at least 28 days of age. That is, the rodent can be 29, 30, 31, 32 or 33 days of age or 28.5 days of age.

The phrase “human immune cell lineages” as used herein refers to cells exhibiting cell surface immune cell markers. The human immune cells can express immune markers, including but not limited to, CD34, CD16, HLA-DR (MHC II), CD14, (monocytes), CD11 c (Dendritic cells), CD11b (Neutrophilic cells), CD19 (B-cells, early stage), CD20 (B-cells, late stage), CD3 (T-cells), CD56 (NK cells) and CD33 (Myloid cells).

The phrase “malignant neoplastic mouse” as used herein refers to an abnormal mass of tissue resulting from abnormal growth or division of cells within a mouse. Neoplastic cell growth surpasses and is not synchronized with normal tissues within its vicinity. Cancer is a form of malignant neoplasm and may or may not form a tumor, i.e. an abnormal mass of tissue.

The phrase “monitoring a side effect of the test substance” as used herein refers to evaluating toxicity, teratogenicity, and other effects a test substance, such as a chemotherapeutic agent, a biologic or a compound or a treatment can have upon a model organism, an in vitro cell culture or other in vitro or in vivo testing systems to determine both efficacy and efficiency but also deleterious or undesirable effects of a test substance. Evaluations can include but are not limited to gross examination of the model system as well as extracting and analyzing blood or blood components, X-ray, dissection and analyses of tissues, organs and cells and cell preparations from the model organism, cell culture or testing system.

The phrase “positively impacts the amount of the human disease in the human disease-infected mouse” as used herein refers to a clinically measurable or quantifiable presence of disease (or infection). The disease would be reduced as measured by tumor size, reduced circulating immune lineage cells, decreased disease (tumor presence) pathology as seen in tissue biopsy and staining and the like as would be known to one of skill in the art.

The phrase “reproduces in the mouse the pathology of disease in the patient from whom the substance is derived” as used herein refers to a PDX mouse having a human disease of the same phenotype and genotype as the human patient from which were extracted the diseased cells that were then transplanted into the mouse.

The phrase “a substance containing a tumor cell” as used herein refers to a liquid containing a tumor cell extracted from at least one of bone marrow or peripheral blood. During extraction of the tumor cell, both plasma and other cells types can be extracted and may remain in the substance containing the tumor cell. Cell types can include but are not limited to tumor cells from lung, breast, gastric and colon cancerous tissues, mononuclear cells, which include leukemic initiator cells, also known as leukemic stem cells, osteoclasts, white blood cells and red blood cells. The tumor cell extracted can be further purified by, for example, passing the tumor cell extract solution through a Ficoll® column to enrich for the tumor cell population. It is the enriched tumor cell population that is contained within the fluidic substance. The substance can be used for injection into a rodent to establish a xenograft of tumor cells in the recipient rodent.

The phrase “selectively expanded” and “selectively expanded cell” as used herein refers to the propagation of human cells by engraftment of human cells into a rodent host. The cells can grow predominantly in the bone marrow, peripheral blood or spleen but can also be detected in the liver and lung and possibly kidney of the engrafted rodent. Testing for CD45+ cells confirmed the progressive take-rate and further supports that the diseased cell was expanded selectively in the human patient derived xenografted (PDX) rodent.

The term “substance” as used herein refers to a solution containing either human tissues or human immune progenitor cells such as mononuclear cells (MNC's), human stem cells, or human stem cells capable of developing into human immune cell lineages such as CD34+ cells or tumor cells derived from a diseased or infected human or mouse. The tumor tissues or tumor cells can be of hemopoiesis origin, or a malignant neoplastic tumor. The tumor can be but is not limited to an epithelial tumor or a tumor of lung, breast, gastro-intestinal including colon origins. The cells can be suspended in PBS, saline or another vehicle as is known to one of skill in the art for injecting intravenously or intraperitoneally cells for engraftment.

### EXAMPLES

### Mice and Engraftment Procedure

Animal experiments were conducted at Crown Bioscience's laboratory, China. The transplantation recipient NOD/SCID mice were purchased from Beijing HFK Bioscience at between 3-4 weeks of age. The Institutional Animal Care and Use Committee (IACUC) of Crown Bioscience approved animal study protocols. All procedures were under sterile conditions at Crown Bioscience's SPF facility and conducted in strict accordance with the Guide for the Care and Use of Laboratory Animals of the National Institutes of Health. CD34+ cells were isolated from a human subject using a CD34 MicroBead Kit® (Miltenyl Biotec Inc., Auburn, Calif.) following the manufacturer's directions.

Patient Samples

All procedures were approved by the Institutional Review Boards (IRB) of the Wuhan Tongji Hospital and with informed consent from the patients.

Mutation Analysis

Hotspot mutation analysis was performed according to the method of Yang, M. et al., supra.

Statistical Analysis

The data for leukemic load were evaluated using Student's t-test for two comparisons, and one-way ANOVA test for multiple comparisons. All data were analyzed using GraphPad Prism 5.0 software (GraphPad Software, Inc. La Jolla, Calif.). Values of P<0.05 were considered to be statistically significant.

Standard Immunohistochemistry, Western Blot and ELISA Analyses

Standard immunohistochemistry (IHC) and Western blot analysis were used to analyze tumor tissues as described before (Yang M. et al. supra).

### EXAMPLES

### Example 1

### Engraftment of human CD34+ cells in NOD/SCID mice

The isolated CD34⁺ cells (2×10̂5 in 100 ul PBS per mouse) were injected into the tail-vein of 3-week old, sublethally irradiated NOD/SCID mice (1.5×10³ rads) also treated with anti-mouse CD122 antibody followed by weekly monitoring for peripheral appearance of Human Immune Cells (CD45⁺) via retro-orbital bleeding and flow analysis. Briefly, ˜50 μL blood was collected from the PDX CD34⁺ mice in EDTA anticoagulation tubes (BD 365974, Becton Dickinson and Co., Franklin Lakes, N.J.). After lysis of red blood cells the white cells were stained with 20 μL mouse anti-human CD45 antibody (Biolegend, San Diego, Calif.) and incubated on ice for 30 minutes in the dark followed by washing with ice cold PBS twice. The cells were resuspended in 150 μL PBS and subjected to FACS analysis using a BD FACSCalibur™ System (Becton Dickinson and Co., Franklin Lakes, N.J.

### Example 2

### Engraftment of Human NSCLC Tissue or Gastric Tumor Tissue in Reconstituted NOD/SCID Mice

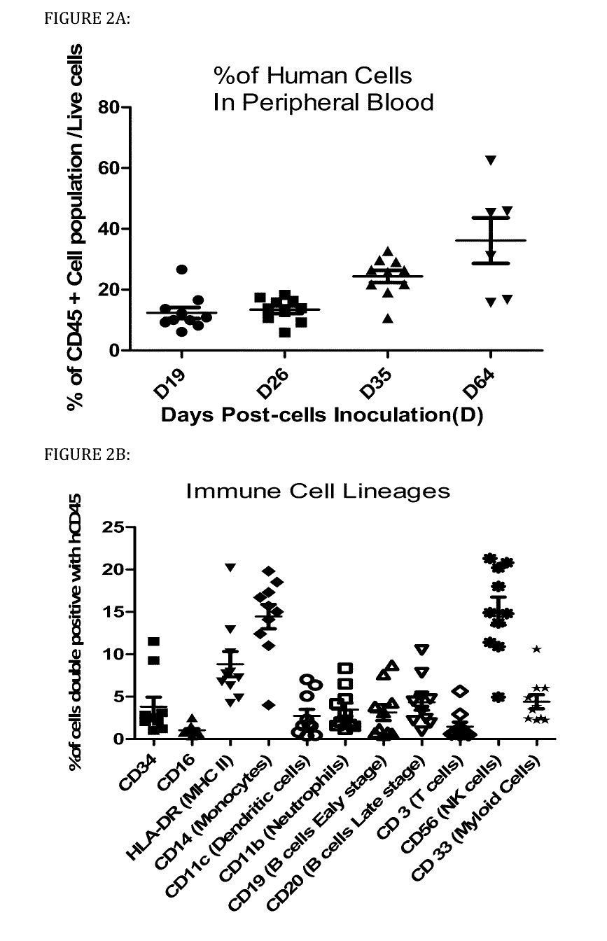

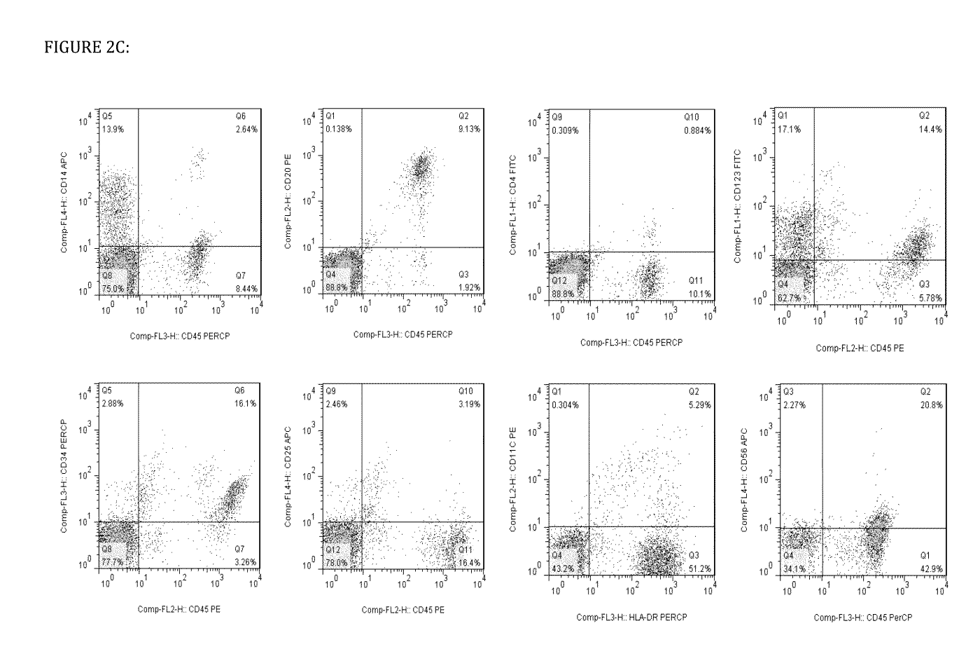

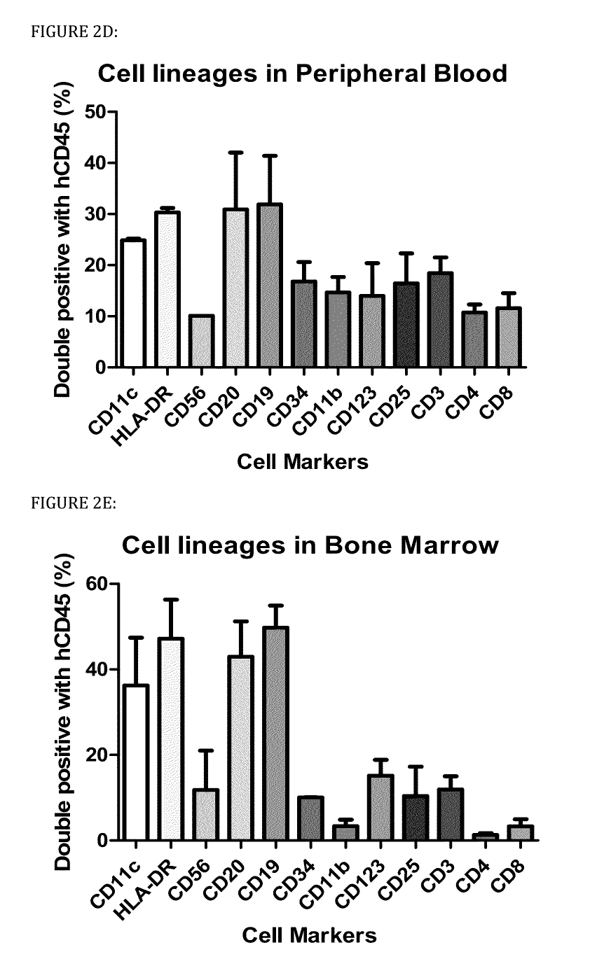

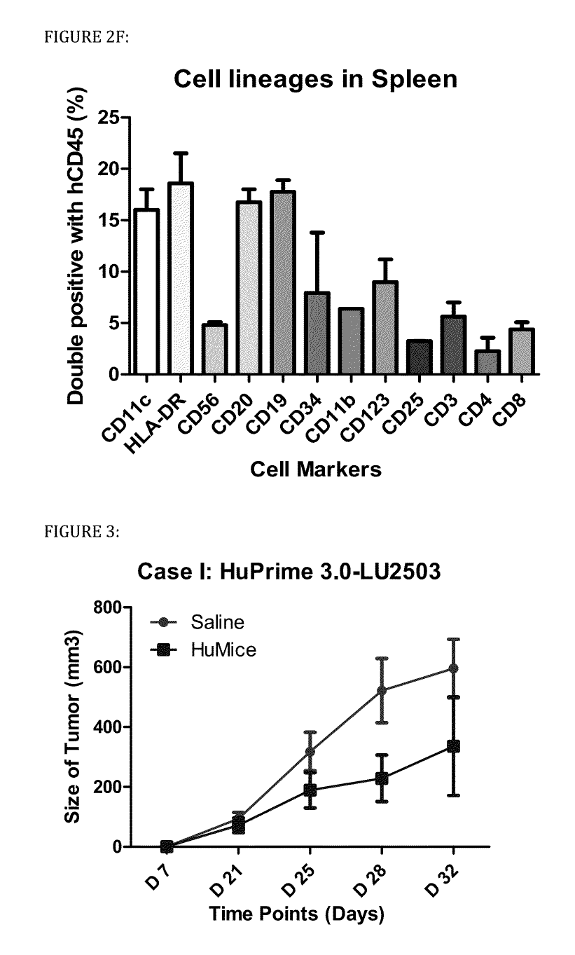

Patient tumor fragments of NSCLC or gastric tumor tissue derived from diseased seed mice were engrafted 2.5 weeks after the CD34+ cell inoculation in order to create patient derived xenograft (PDX) models. Solid tumor sample fragments from seed mice inoculated with selected primary human lung or gastric cancer tissues were harvested and used for inoculation into mice. Each mouse was inoculated subcutaneously at the right flank with one tumor fragment (2-3 mm in diameter) for tumor development. The treatments were started when mean tumor size reached approximately 100-150 mm³ The transplanted animals were monitored by weekly bleed and FACS analysis for hCD45⁺ cells in peripheral blood. All five transplanted mice (P0) showed tumor cell growth in peripheral blood (FIG. 2A) with relative long latency (5 months). LU250 and LU0387 mice ultimately developed full-blown NSCLC with different human immune cell lineages as seen by FACS analysis of PB, BM and spleen (FIGS. 2B-2F) accompanied with typical symptoms of body weight loss and tumor growth.

### Example 3

### Characterizations of LU250 & LU0387-Mice

High Engraftment Levels in Different Animal Organs.

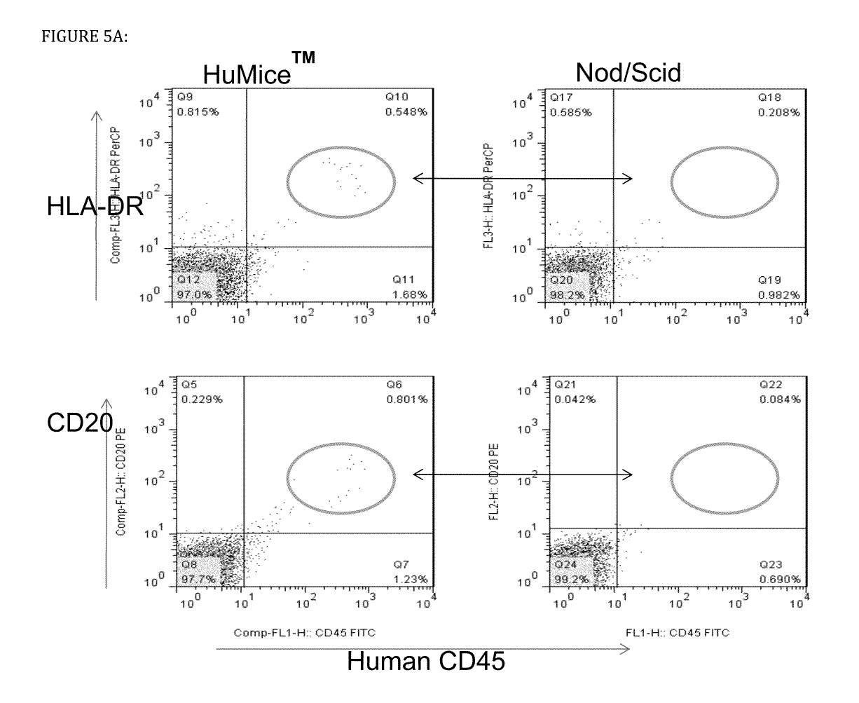

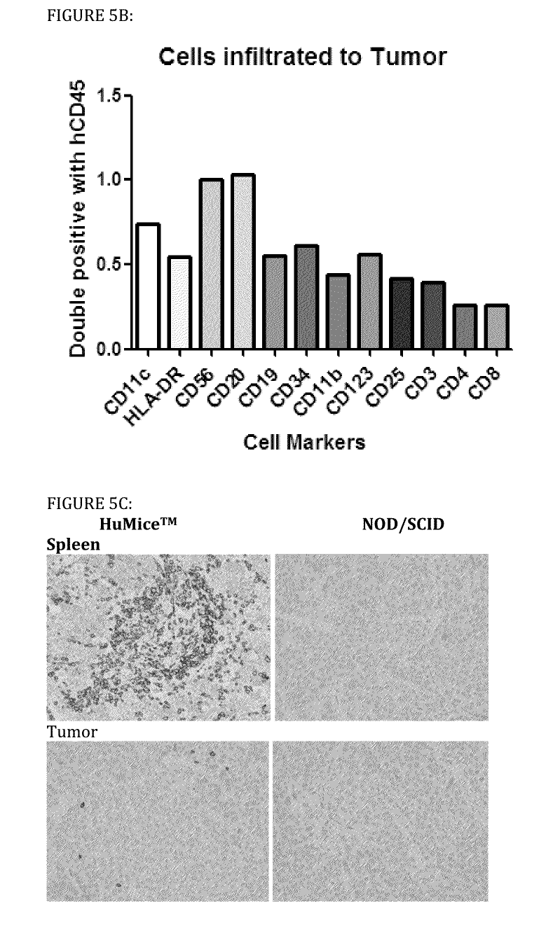

Not only were the tumor cells of patients LU250 & LU0387 found to have a good rate of engraftment, but they also displayed infiltration of human immune cells (FIG. 5A) and different human immune system cell lineage loads as measured by flow cytometry monitoring of hCD45⁺ cells in the developed tumor (FIG. 5B). In addition, immunohistochemistry (IHC) was also used to confirm the infiltration of tumor cells into different organs, including spleen and the PDX tumor, as shown in FIG. 5C.

### Example 4

### Evidence of NK Cell Activities in Reconstituted Mice

HuMice™ and NOD/SCID mice were engrafted with cells from a gastric tumor patient followed by treatment with either Herceptin or Cisplatin. We tested Herceptin and Cisplatin anti-tumor activity against GA 0055 tumor, by subjecting GA 0055-mice to a 5-day-on/2-day-off Herceptin or Cisplatin dosing regimen, starting 15 days post-engraftment (Table 3)

TABLE 3
Herceptin or Cisplatin dosing regimen

| | | Dose | Number of | Pre- | Dosing | Dosing |
|---|---|---|---|---|---|---|
| Group | Treatment | (mg/kg) | Mice | treatment | Route | Schedule |
| 1 | Vehicle | — | 4 | | — | — |
| 2 | Herceptin | 6 | 4 | Human Stem | i.v | D1, 4/wk |
| | | | | cells | | |
| 3 | Cisplatin | 5 | 4 | | i.P | Weekly |
| 4 | Vehicle | — | 4 | | — | — |
| 5 | Herceptin | 6 | 4 | — | i.v | D1, 4/wk |
| 6 | Cisplatin | 5 | 4 | | i.p | Weekly |

FIGS. 6A-6C illustrate the presence of GA 0055 tumors and tumor expression levels and the data as shown in FIGS. 7A-7D illustrate that in the presence of NK cells NK cell activities were participating in Herceptin treatment but were not involved in treatment with a chemotherapeutic agent, i.e. Cisplatin.

While various embodiments of the present invention have been described in detail, it is apparent that modifications, adaptations and equivalents of those embodiments will occur to those skilled in the art. It is to be expressly understood, however, that such modifications and adaptations are within the scope of the present invention and are intended to be encompassed herein, as set forth in the following claims.

### REFERENCES

Clynes, R A; Towers, T L; Presta, L G; Ravetch, T V. “Inhibitory Fc receptors modulate in vivo cytoxicity against tumor targets”. 2000, Nat. Med. 6(4):443-6. doi:10.1038/74704.
Ding, L., et al. Genome remodelling in a basal-like breast cancer metastasis and xenograft. 2010 Nature 464:999-1005.
Marangoni, E., et al. A new model of patient tumor-derived breast cancer xenografts for preclinical assays. 2007 Clin Cancer Res 13:3989-3998.
3. Yang, M., Baoen Shan, Qiaoxia Li, Xiaoming Song, Jianyun Deng, Jie Cai, Likun Zhangl, Junjie Lu, Zhenjian Du, Taiping Chen, Jean-Pierre Wery, Yiyou Chen and Qixiang Li. Overcoming erlotinib resistance with tailored treatment regimen in patient derived xenografts from naïve Asian NSCLC patients. Int J Cancer September 5. doi: 10.1002/ijc.27813. Epub ahead of print] (2012).

### ISSUED PATENTS AND PUBLISHED PATENT APPLICATIONS

U.S. Pat. No. 8,569,573; issued Oct. 29, 2013 Grompe, et al.
U.S. Pat. No. 8,541,646; issued Sep. 24, 2013 Stevens, Sean et al.
U.S. Ser. No. 14/053,182; published Oct. 14, 2013 Stevens, Sean et al.
U.S. Ser. No. 13/673,190, published 5/16/13Giovanella, Beppino

It is claimed:

1. A method for screening for a therapeutic agent for a diseased human, the method comprising:
a. administering an agent to a first immunocompromised NOD/SCID mouse, wherein the mouse
i) is immunodeficient for a mouse immune system,
ii) is engrafted with human stem cells containing human CD34+ cells, wherein the CD34+ cells are isolated from fetal liver, peripheral blood or umbilical cord blood cells, and
iii) is engrafted with a substance derived from a diseased human containing a diseased cell or tissue derived from a human diseased patient or is engrafted with a substance derived from a second immunocompromised NOD/SCID mouse engrafted with a substance derived from a diseased human containing a diseased cell derived from a human diseased patient; and
b. determining whether the agent positively impacts the amount of the human disease in the human disease-infected mouse.

2. The method according to claim 1, wherein the mouse is immunocompetent for a human immune system.

3. The method according to claim 1, wherein the mouse is displays the histopathology of the disease of the human disease patient.

4. The method according to claim 1, wherein the mouse displays the genetic profile of the diseased cell derived from the human disease patient.

5. The method according to claim 1, wherein the agent is a drug, or a biologic.

6. The method according to claim 1, wherein the human disease is selected from a malignant neoplasia and an infection.

7. The method according to claim 1, wherein the diseased cell derived from a human diseased patient is selected from an epithelial cell, a bacterium, a parasite, a virus, a piron or a microbacterium.

8. The method according to claim 1, wherein the amount of the human disease impacted by the agent is determined by monitoring at least one of: tumor size, tumor pathology, human cell markers within the tumor, prolonged survival, no disease relapse, and immune response and combinations thereof.

9. The method according to claim 5, wherein the biologic is selected from an antibody, a vaccine, an allergenic, a somatic cell, a gene therapy, a tissue, a recombinant therapeutic protein or a living cell used as a therapeutic.

10. The method according to claim 6, wherein the malignant neoplasia is selected from a non-small cell lung cancer, a breast cancer, a gastric cancer, and a colon cancer.

11. The method according to claim 8, wherein the tumor pathology is determined by staining for hematopoiesis cells.

12. The method according to claim 1, wherein the hematopeiesis cells are selected from lymphocytes, dentritic cells, macrophage cells, monocytes, MHC-II cells, pre-B cells, B-cells, T-cells, and natural killer cells.

13. The method according to claim 12, wherein the tumor pathology indicates the presence of a human tumor infiltrate lymphocyte (TIL) cell.

14. The method according to claim 12, wherein the tumor pathology indicates the presence of an inflammatory cell.

15. The method according to claim 14, wherein the inflammatory cell is at least one of a CD4, CD11b, CD14 and CD33 cell and combinations thereof.

16. The method according to claim 8, wherein the human cell markers within the tumor are at least one CD11 c in Dentritic Cells; HLA-DR in MHC-II Cells; CD19 in Pre-B cells; CD20 in B− Cells; CD4 in T− Cells; CD56 in NK Cells; CD123 in Cells with IL-3 receptor; and CD25, wherein the cell markers are detectable markers of lymphocyte activation for many immune cells.

17. The method according to claim 7, wherein the epithelial cell is from a non-small cell lung cancer, a breast cancer, a gastric cancer or a colon cancer.

18. The method according to claim 1, wherein the second immunocompromised NOD/SCID mouse is engrafted with a substance derived from a third or a fourth or fifth or sixth diseased mouse engrafted with a substance containing a diseased cell derived from the same or a different human diseased patient or from a second different diseased mouse engrafted with a substance derived from a different diseased mouse engrafted with a substance containing a diseased cell derived from an engrafted diseased mouse having the human disease.

19. A method of selecting or optimizing a method of treating a patient from whom tumor cells in the peripheral blood are derived from an immunocompromised NOD/SCID mouse engrafted with human stem cells containing human CD34+ cells, wherein the CD34+ cells are isolated from fetal liver, peripheral blood or umbilical cord blood cells and engrafted with a substance derived from:
i) a diseased human containing a diseased cell derived from a human diseased patient, or
ii) a diseased mouse containing a diseased cell derived from at least a second or a third immunocompromised NOD/SCID diseased mouse engrafted with a substance derived from a second diseased mouse containing a diseased cell derived from a human diseased patient,
a) providing the mouse with a treatment for the tumor, and
b) assessing an improvement and/or a side effect caused by the treatment of the tumor in the mouse.

20. An immunocompromised NOD/SCID mouse comprising a functioning human immune system and a replicate pathology of a human disease, wherein the mouse is a xenografted mouse.
